SEQUENCE LISTING (96 sequences)

SEQ ID NO: 1 - DNA, 25 bp, Bos taurus
  source 1..25: mol_type = genomic DNA
tttccttctgtagatgttaactggt

SEQ ID NO: 2 - DNA, 23 bp, Bos taurus
  source 1..23: mol_type = genomic DNA
acggacaataaactgtaaatttc

SEQ ID NO: 3 - DNA, 26 bp, Bos taurus
  source 1..26: mol_type = genomic DNA
cacttgtatgtatttcagaagttttc

SEQ ID NO: 4 - DNA, 20 bp, Bos taurus
  source 1..20: mol_type = genomic DNA
aggatcacaaacaatgccct

SEQ ID NO: 5 - DNA, 26 bp, Bos taurus
  source 1..26: mol_type = genomic DNA
tctgaaggaattgaaaatgtctacca

SEQ ID NO: 6 - DNA, 24 bp, Bos taurus
  source 1..24: mol_type = genomic DNA
acttctacaattagcgattaactg

SEQ ID NO: 7 - DNA, 24 bp, Bos taurus
  source 1..24: mol_type = genomic DNA
tcagaggagaatgtctagtttaga

SEQ ID NO: 8 - DNA, 18 bp, Bos taurus
  source 1..18: mol_type = genomic DNA
tcagtgtgacggagctgc

SEQ ID NO: 9 - DNA, 20 bp, Bos taurus
  source 1..20: mol_type = genomic DNA
tcatgagaaatcagcccaca

SEQ ID NO: 10 - DNA, 24 bp, Bos taurus
  source 1..24: mol_type = genomic DNA
acctgctgtgtaatgtatttaaac

SEQ ID NO: 11 - DNA, 24 bp, Bos taurus
  source 1..24: mol_type = genomic DNA
aggtttaagggaatatttgcacct

SEQ ID NO: 12 - DNA, 24 bp, Bos taurus
  source 1..24: mol_type = genomic DNA
tttgaataactgtacagggaattc

SEQ ID NO: 13 - DNA, 25 bp, Bos taurus
  source 1..25: mol_type = genomic DNA
gcttaaagttctaaaccaatcaaca

SEQ ID NO: 14 - DNA, 25 bp, Bos taurus
  source 1..25: mol_type = genomic DNA
agctcttctaaaacaagtaaagcca

SEQ ID NO: 15 - DNA, 22 bp, Bos taurus
  source 1..22: mol_type = genomic DNA
ccaagaaccactgtgataggag

SEQ ID NO: 16 - DNA, 22 bp, Bos taurus
  source 1..22: mol_type = genomic DNA
taagaaaagagggaacaagact

SEQ ID NO: 17 - DNA, 21 bp, Bos taurus
  source 1..21: mol_type = genomic DNA
atgtggcttcctgtattccct

SEQ ID NO: 18 - DNA, 26 bp, Bos taurus
  source 1..26: mol_type = genomic DNA
gttcagcaaataatttatagagaacc

SEQ ID NO: 19 - DNA, 20 bp, Bos taurus
  source 1..20: mol_type = genomic DNA
ctggcccaaactcatcacac

SEQ ID NO: 20 - DNA, 21 bp, Bos taurus
  source 1..21: mol_type = genomic DNA
acaaaccaaccaccaaacaga

SEQ ID NO: 21 - DNA, 20 bp, Bos taurus
  source 1..20: mol_type = genomic DNA
tgagtttcagagagggccag

SEQ ID NO: 22 - DNA, 20 bp, Bos taurus
  source 1..20: mol_type = genomic DNA
gccacttgatgccataggtc

SEQ ID NO: 23 - DNA, 20 bp, Bos taurus
  source 1..20: mol_type = genomic DNA
agaaagccatacccagggag

SEQ ID NO: 24 - DNA, 21 bp, Bos taurus
  source 1..21: mol_type = genomic DNA
agacaacagtgaagttcaggc

SEQ ID NO: 25 - DNA, 20 bp, Bos taurus
  source 1..20: mol_type = genomic DNA
cccatgtatgtgtagctggc

SEQ ID NO: 26 - DNA, 21 bp, Bos taurus
  source 1..21: mol_type = genomic DNA
ctgaggaagacagggagaagg

SEQ ID NO: 27 - DNA, 24 bp, Bos taurus
  source 1..24: mol_type = genomic DNA
acataagtacatatctactggcct

SEQ ID NO: 28 - DNA, 22 bp, Bos taurus
  source 1..22: mol_type = genomic DNA
tgagaaccacttaagatagggt

SEQ ID NO: 29 - DNA, 26 bp, Bos taurus
  source 1..26: mol_type = genomic DNA
tactaattgtatatcttgctgctcag

SEQ ID NO: 30 - DNA, 19 bp, Bos taurus
  source 1..19: mol_type = genomic DNA
ctgccttccctcctcagtc

SEQ ID NO: 31 - DNA, 22 bp, Bos taurus
  source 1..22: mol_type = genomic DNA
aggtattatgagttgtgtgggt

SEQ ID NO: 32 - DNA, 24 bp, Bos taurus
  source 1..24: mol_type = genomic DNA
acatcctcttacctaatctgagct

SEQ ID NO: 33 - DNA, 21 bp, Bos taurus
  source 1..21: mol_type = genomic DNA
ggaaaccctatgagccagagt

SEQ ID NO: 34 - DNA, 18 bp, Bos taurus
  source 1..18: mol_type = genomic DNA
tccctcagcccctttcag

SEQ ID NO: 35 - DNA, 20 bp, Bos taurus
  source 1..20: mol_type = genomic DNA
gggagtggagagtggattgg

SEQ ID NO: 36 - DNA, 20 bp, Bos taurus
  source 1..20: mol_type = genomic DNA
tgccccttttgtacagatgg

SEQ ID NO: 37 - DNA, 20 bp, Bos taurus
  source 1..20: mol_type = genomic DNA
attctcttgacttgcaggcg

SEQ ID NO: 38 - DNA, 25 bp, Bos taurus
  source 1..25: mol_type = genomic DNA
gtcaataactcaataaaagcactga

SEQ ID NO: 39 - DNA, 19 bp, Bos taurus
  source 1..19: mol_type = genomic DNA
tgggttcttgggtccagag

SEQ ID NO: 40 - DNA, 20 bp, Bos taurus
  source 1..20: mol_type = genomic DNA
aggtcccagtctcctccttc

SEQ ID NO: 41 - DNA, 20 bp, Bos taurus
  source 1..20: mol_type = genomic DNA
gactgcctggatctgagagg

SEQ ID NO: 42 - DNA, 20 bp, Bos taurus
  source 1..20: mol_type = genomic DNA
caacgggacacttgcctttc

SEQ ID NO: 43 - DNA, 20 bp, Bos taurus
  source 1..20: mol_type = genomic DNA
atgtctagccctcactccca

SEQ ID NO: 44 - DNA, 21 bp, Bos taurus
  source 1..21: mol_type = genomic DNA
cgtgatcaaggcagaactaca

SEQ ID NO: 45 - DNA, 20 bp, Bos taurus
  source 1..20: mol_type = genomic DNA
aagagcagctggtttcctat

SEQ ID NO: 46 - DNA, 22 bp, Bos taurus
  source 1..22: mol_type = genomic DNA
aggggagaagatatgcagacag

SEQ ID NO: 47 - DNA, 20 bp, Bos taurus
  source 1..20: mol_type = genomic DNA
gtcctcatcagcctcgtcat

SEQ ID NO: 48 - DNA, 20 bp, Bos taurus
  source 1..20: mol_type = genomic DNA
atatccaccctgcaaaccca

SEQ ID NO: 49 - DNA, 20 bp, Bos taurus
  source 1..20: mol_type = genomic DNA
agtgctatcatgtgccttga

SEQ ID NO: 50 - DNA, 21 bp, Bos taurus
  source 1..21: mol_type = genomic DNA
acacagactttaaagcagcct

SEQ ID NO: 51 - DNA, 18 bp, Bos taurus
  source 1..18: mol_type = genomic DNA
gaggggaggggagggttg

SEQ ID NO: 52 - DNA, 20 bp, Bos taurus
  source 1..20: mol_type = genomic DNA
gagaattcaggggcccttct

SEQ ID NO: 53 - DNA, 24 bp, Bos taurus
  source 1..24: mol_type = genomic DNA
ggataatctttagcaatcagaggc

SEQ ID NO: 54 - DNA, 20 bp, Bos taurus
  source 1..20: mol_type = genomic DNA
tctccccgcttgtaacagtt

SEQ ID NO: 55 - DNA, 19 bp, Bos taurus
  source 1..19: mol_type = genomic DNA
aaaacttgctgccagggaa

SEQ ID NO: 56 - DNA, 18 bp, Bos taurus
  source 1..18: mol_type = genomic DNA
cacgaggcacatatcagt

SEQ ID NO: 57 - DNA, 20 bp, Bos taurus
  source 1..20: mol_type = genomic DNA
cctgttacctgggcttcatg

SEQ ID NO: 58 - DNA, 22 bp, Bos taurus
  source 1..22: mol_type = genomic DNA
acgactcggcatagatgatttg

SEQ ID NO: 59 - DNA, 22 bp, Bos taurus
  source 1..22: mol_type = genomic DNA
cctctctttcaggccaaaatcc

SEQ ID NO: 60 - DNA, 26 bp, Bos taurus
  source 1..26: mol_type = genomic DNA
ctattctattttcctcaagtatctgc

SEQ ID NO: 61 - DNA, 21 bp, Bos taurus
  source 1..21: mol_type = genomic DNA
attcagattgatggtccagca

SEQ ID NO: 62 - DNA, 21 bp, Bos taurus
  source 1..21: mol_type = genomic DNA
agtggtgatctcaaagaggct

SEQ ID NO: 63 - DNA, 22 bp, Bos taurus
  source 1..22: mol_type = genomic DNA
taactgcatcgttaaactggct

SEQ ID NO: 64 - DNA, 19 bp, Bos taurus
  source 1..19: mol_type = genomic DNA
cttctggagctcacccacc

SEQ ID NO: 65 - DNA, 19 bp, Bos taurus
  source 1..19: mol_type = genomic DNA
cactcagacgtccccaacc

SEQ ID NO: 66 - DNA, 20 bp, Bos taurus
  source 1..20: mol_type = genomic DNA
gggttcggcaagattcaagt

SEQ ID NO: 67 - DNA, 18 bp, Bos taurus
  source 1..18: mol_type = genomic DNA
gaggctgccaggtgtcac

SEQ ID NO: 68 - DNA, 20 bp, Bos taurus
  source 1..20: mol_type = genomic DNA
tgaaaaggtagcccaggtgt

SEQ ID NO: 69 - DNA, 19 bp, Bos taurus
  source 1..19: mol_type = genomic DNA
gttttgccttccacccacc

SEQ ID NO: 70 - DNA, 23 bp, Bos taurus
  source 1..23: mol_type = genomic DNA
tctccaaagcaaacaagttagtg

SEQ ID NO: 71 - DNA, 17 bp, Bos taurus
  source 1..17: mol_type = genomic DNA
ccaacctgttcagcaca

SEQ ID NO: 72 - DNA, 20 bp, Bos taurus
  source 1..20: mol_type = genomic DNA
agatcttccccaacagtact

SEQ ID NO: 73 - DNA, 20 bp, Bos taurus
  source 1..20: mol_type = genomic DNA
ggaagtcccttgtgctcatc

SEQ ID NO: 74 - DNA, 20 bp, Bos taurus
  source 1..20: mol_type = genomic DNA
aagtagaccacgcccctttc

SEQ ID NO: 75 - DNA, 23 bp, Bos taurus
  source 1..23: mol_type = genomic DNA
aaagtaataaggcttgccttcca

SEQ ID NO: 76 - DNA, 23 bp, Bos taurus
  source 1..23: mol_type = genomic DNA
tgagaaatattgactacacgccc

SEQ ID NO: 77 - DNA, 21 bp, Bos taurus
  source 1..21: mol_type = genomic DNA
aaggggctcataagataaagc

SEQ ID NO: 78 - DNA, 21 bp, Bos taurus
  source 1..21: mol_type = genomic DNA
ccagagatggggcatagaact

SEQ ID NO: 79 - DNA, 17 bp, Bos taurus
  source 1..17: mol_type = genomic DNA
gtgcctggctctgtaca

SEQ ID NO: 80 - DNA, 16 bp, Bos taurus
  source 1..16: mol_type = genomic DNA
tgccgacctcacgtgg

SEQ ID NO: 81 - DNA, 20 bp, Bos taurus
  source 1..20: mol_type = genomic DNA
gtccatccatgttgctgcaa

SEQ ID NO: 82 - DNA, 18 bp, Bos taurus
  source 1..18: mol_type = genomic DNA
tactggccaagaacacct

SEQ ID NO: 83 - DNA, 23 bp, Bos taurus
  source 1..23: mol_type = genomic DNA
attatgttttaatgcactgctgt

SEQ ID NO: 84 - DNA, 20 bp, Bos taurus
  source 1..20: mol_type = genomic DNA
ttggcattgggaaagggaaa

SEQ ID NO: 85 - DNA, 20 bp, Bos taurus
  source 1..20: mol_type = genomic DNA
tgtgggttgtagtagcagca

SEQ ID NO: 86 - DNA, 20 bp, Bos taurus
  source 1..20: mol_type = genomic DNA
acccagtcacattcagagct

SEQ ID NO: 87 - DNA, 22 bp, Bos taurus
  source 1..22: mol_type = genomic DNA
actaaaatacaaatgcaggcac

SEQ ID NO: 88 - DNA, 26 bp, Bos taurus
  source 1..26: mol_type = genomic DNA
ggtaccctgtattctttcaaatgagt

SEQ ID NO: 89 - DNA, 22 bp, Bos taurus
  source 1..22: mol_type = genomic DNA
tgctgcagattctttacttgct

SEQ ID NO: 90 - DNA, 21 bp, Bos taurus
  source 1..21: mol_type = genomic DNA
tctctagcccatgtcatgact

SEQ ID NO: 91 - DNA, 25 bp, Bos taurus
  source 1..25: mol_type = genomic DNA
tcttttcatccttaccatagctagg

SEQ ID NO: 92 - DNA, 20 bp, Bos taurus
  source 1..20: mol_type = genomic DNA
ttgcagatgtgatcaaggct

SEQ ID NO: 93 - DNA, 24 bp, Bos taurus
  source 1..24: mol_type = genomic DNA
aggattgtatttaaggttgtctga

SEQ ID NO: 94 - DNA, 20 bp, Bos taurus
  source 1..20: mol_type = genomic DNA
tgatgcccacaataccaggt

SEQ ID NO: 95 - DNA, 22 bp, Bos taurus
  source 1..22: mol_type = genomic DNA
caggcagatggttttaacacac

SEQ ID NO: 96 - DNA, 26 bp, Bos taurus
  source 1..26: mol_type = genomic DNA
agataatatagactgtagtgctgggt

(19) United States
(12) Patent Application Publication
(10) Pub. No.: US 2026/0218272 A1
(43) Pub. Date: Jul. 30, 2026

(54) METHODS AND SYSTEMS FOR PROCESSING GENETIC SAMPLES TO DETERMINE IDENTITY OR DETECT CONTAMINATION

(71) Applicant: ABS Global, Inc., DeForest, WI (US)

(72) Inventors: Jennifer Dzelil, Waunakee, WI (US); Matthew Campbell, Waunakee, WI (US); Steve Rounsley, DeForest, WI (US); Patrice Linel, DeForest, WI (US)

(21) Appl. No.: 19/419,534

(22) Filed: Dec. 15, 2025

Related U.S. Application Data
(63) Continuation of application No. 17/507,662, filed on Oct. 21, 2021, now Pat. No. 12,497,648.
(60) Provisional application No. 63/094,750, filed on Oct. 21, 2020.

(51) Int. Cl. C12Q 1/6809; C12Q 1/686; C12Q 1/6869; C12Q 1/6876

(52) U.S. Cl. CPC C12Q 1/6809; C12Q 1/686; C12Q 1/6869; C12Q 1/6876

(57) ABSTRACT

Methods and systems for processing semen samples from straws for determining genetic identity, testing the purity of the sample, detecting errors or contamination, calculating an amount of contamination, and determining the identity of the contaminant. The methods herein can detect low levels of contamination, such as contamination of about 0.5%, 1%, 2%, etc.

### CROSS-REFERENCES TO RELATED APPLICATIONS

This application is a continuation and claims benefit of U.S. Provisional application Ser. No. 17/507,662 filed Oct. 21, 2021, which is a non-provisional and claims benefit of U.S. Provisional Application No. 63/094,750 filed Oct. 21, 2020, the specification(s) of which is/are incorporated herein in their entirety by reference.

### REFERENCE TO AN ELECTRONIC SEQUENCE LISTING

The contents of the electronic sequence listing (GENUS_19_03_CON_Sequence_Listing.xml; Size: 84,829 bytes; and Date of Creation: Dec. 15, 2025) is herein incorporated by reference in its entirety.

### BACKGROUND OF THE INVENTION

### Field of the Invention

The present invention relates to methods for amplification, sequencing, and analyzing genetic samples, more particularly to methods and systems for processing genetic samples, e.g., sperm samples, for verifying or determining identity of the samples, detecting genetic contamination therein, and/or detecting mislabeling of samples.

### Background Art

When preparing animal semen straws, it is possible to incorrectly label the straw or accidentally introduce unwanted genetic material. For example, samples may first be split into multiple aliquots, run through machines for sex-selection, and then subsequently recombined. It is possible during the recombination step, or other steps, to introduce contamination or mix samples from different bulls, resulting in a calf being born with the incorrect sire.

Existing methods for evaluating genetic purity in corn, soy, or other such samples (e.g., current microarray or chip assays) primarily focus on determining if an evaluated sample is of a sufficient purity or low enough contamination level to be usable for additional downstream analysis. Existing methods also functionally measure one allele per line and may only be able to identify the presence of the given lines in a sample. Without wishing to limit the present invention to any theory or mechanism, it is believed that these methods are generally insufficient for determining if a sample comprises only a desired set of genetic material.

However, it was surprisingly discovered that multiplex sequencing methods could be used to detect low levels of contamination in straw samples, such as levels of contamination as low as 1% (or less than 1%), 2%, 3%, 4%, 5%, etc. This was surprising, since multiplex sequencing methods generally rely on uniform samples and the straw samples are non-uniform since they contain dead sperm, UV-irradiated sperm, etc.

### BRIEF SUMMARY OF THE INVENTION

The present invention features methods and systems for processing genetic samples, e.g., sperm samples (e.g., samples from semen straws). The methods and systems herein allow for the verification or determination of the identity of the sample. For example, the methods and systems herein allow for determining/confirming that the genetic content in the sample matches the reference animal (e.g., confirm or determine if there is a 100% genetic match). The methods and systems herein also test the purity of samples so as to detect possible mixtures or contamination. In certain embodiments, the methods and systems herein also feature steps for determining the identity of the contaminant or the origin of the genetic material (e.g., in the case of a mislabeled sample).

Without wishing to limit the present invention to any theory or mechanism, it is believed that the methods and systems herein are advantageous because they allow for easy, fast, and highly sensitive identity confirmation and/or detection of contamination. Multiplexing helps decrease the cost and help with uniformity.

The methods and systems herein help determine (to an extent) that a tested sample comprises only a desired set of genetic material. By determining genetic purity of the semen straws to such an extent, it is possible to ensure to a greater degree that the straws contain the sperm they claim, thus avoiding costly errors. Also, certain jurisdictions require high purity standards, and the methods herein can help ensure certain sperm straws can be sold.

As previously discussed, when preparing samples of genetic material, it is possible that the samples may be incorrectly labeled or that the labels may be incorrectly read. Further, mixing or contamination may occur when splitting or separating samples and later recombining the samples, or contamination may occur when processing genetic samples for genotyping. The methods and systems herein can be used to help address the aforementioned problems that may arise with respect to the processing, handling, and quality control of genetic material.

Without wishing to limit the present invention to any theory or mechanism, it is believed that the methods and systems herein are advantageous because they improve on existing methods that were directed to quality control of sequencing data sets, and the methods and systems herein are advantageous in the field of semen processing. Existing methods generally checked for contamination or sample switching in sequencing data to determine if the sequencing data was suitable. The present invention provides an approach that may identify contamination in the sequencing run to both confirm the identity of the sample being tested (e.g., determine if there is a match between the expected individual in the sample and the actual individual identified in sample), and identify any contamination in the genetic sample (e.g., semen straw), both of which are not provided by existing methods.

Briefly, the test sample, e.g., genetic sample (e.g., isolated or extracted DNA from the test sample), is subjected to amplification (e.g., PCR amplification) using a pool of SNP primer pairs, wherein each SNP primer pair flanks a unique locus that contains a single SNP defining a first allele and a second allele. The amplification (e.g., PCR amplification) produces amplicons for each SNP allele, thus generating a pool of SNP amplicons. The methods further comprise subjecting the pool of SNP amplicons to sequencing (e.g., next-generation sequencing (NGS)), wherein sequencing provides a nucleotide sequence for each amplicon in the pool of SNP amplicons.

The results from the sequencing are provided to an analysis system, e.g., a computer-based system for compiling and/or organizing and/or performing mathematical or statistical operations on the information obtained from sequencing. For example, the analysis system may calculate the frequency of the first allele and the second allele for each SNP. In certain embodiments, the system compares the sequences and/or allele frequencies for each SNP to the corresponding SNPs of at least one reference sequence (e.g., a library sequence, a sequence from a known individual, e.g., a known bull, etc.). A subset of the corresponding SNPs of the reference sequence (e.g., library sequence, sequence from a reference individual, e.g., known bull, etc.) are expected=homozygous SNPs. In certain embodiments, the method comprises calculating the frequency of a non-matching allele for each SNP in the test sample corresponding to each expected homozygous SNP in the reference sequence. In some embodiments, particular calculations are based on the frequency of a non-matching allele for each expected homozygous SNP and the number of SNPs with a particular frequency of non-matching alleles, allowing for the determination of a rate of contamination by one or more genetically distinct individuals. The analysis helps to detect a genetic match (e.g., confirm identity), to detect a mixture, to determine the identity of the genetic sample, and/or determine origin of contaminating material, etc.

The genetic samples referred to in methods and systems herein comprise sperm samples, e.g., sperm samples obtained from bulls. However, the present invention is not limited to sperm samples, nor is the present invention limited to samples obtained from bulls. As previously discussed, the sperm samples may be stored in straws. In certain embodiments, the sperm sample may comprise live sperm and sperm having been subjected to UV irradiation. In some embodiments, the sperm sample comprises live sperm and dead sperm. In some embodiments, the sperm sample has been subjected to a machine for determining the sex of animal.

The methods herein are described as automated, multiplex methods. However, the present invention is not limited to automated and multi-sample applications. Multiplex assays are well known to one of ordinary skill in the art, wherein at least 2 samples are subjected to the method simultaneously, e.g., at least 2, at least 6, at least 12, at least 24, at least 48, at least 96, etc. samples are subjected to the method simultaneously.

The methods described herein may feature additional steps where samples are re-tested to confirm results, e.g., a non-100% match sample or one labeled as a potential mixture may be retested to confirm it is contaminated (or a swap) instead of being automatically discarded. In certain embodiments, samples are discarded after having been tested at least twice and two or more times have shown to be a non-100% match.

The methods herein may also feature steps for analyzing the sample for quantity. In certain embodiments, if the sample is determined to have insufficient quantity, another sample may be obtained from the source (e.g., sperm straw).

The present invention also includes providing results from the amplification and sequencing to an integrated analysis system (e.g., application) with a user interface.

The analysis system (e.g., application) can display results via the user interface. Examples of results may include but are not limited to: SNP read results from sequencing, summaries of the sequencing results, errors or alerts, etc. For example, the analysis system may be programmed to show an alert (e.g., via the user interface) in certain circumstances, e.g., if there is insufficient genetic material for analysis, if there is suspected contamination, if there is not a genetic match, etc. The user (e.g., technician) may review the results and determine if a manual review of the data and/or a rerun of the sample is required based on the visual data shown on the user interface (e.g., the visual data showing the SNP read results from the sequencing).

The present invention provides a method of processing extracted DNA from a test sample. In certain embodiments, the method comprises subjecting the extracted DNA from the test sample to nucleotide amplification using a pool of SNP primer pairs, each SNP primer pair flanking a unique locus that contains a single target SNP defining a first allele and a second allele, the nucleotide amplification produces amplicons for each SNP primer pair generating a pool of SNP amplicons; subjecting the pool of SNP amplicons to sequencing to generate a nucleotide sequence for each amplicon in the pool; and calculating a frequency of the first allele and the second allele for each SNP. The frequencies of the first alleles and second alleles for each SNP may be compared. For example, in certain embodiments, the frequencies of the first alleles and the second alleles for each SNP in a subset of the target SNPs to a reference sequence, wherein the subset of target SNPs is a group of SNPs expected to be homozygous. If the frequencies of the first alleles and second alleles for each SNP in the subset of target SNPs are an exact match to the corresponding SNPs in the reference sequence, then the test sample is the same as that of the reference sequence, e.g., there is a genetic match. If the frequencies of the first alleles and second alleles for each SNP in the subset of SNPs are not exact matches to the corresponding SNPs in the reference sequence, then a frequency of non-matching alleles is calculated for the SNPs in the subset of SNPs (e.g., the SNPs of the subset that are not exact matches). If the frequency of the non-matching alleles for a particular SNP is above a predetermined threshold (e.g., a “non-matching threshold”), then the particular SNP is considered to be a contaminating SNP (e.g., a potential indication of a mixture or error). If the number of contaminating SNPs is above a predetermined threshold (e.g., a “contaminating SNP threshold”), then the sample is identified as contaminated (e.g., the sample is considered to be a potential mixture or contain errors). In some embodiments, the sample is determined to be a mislabeled sample or a swap.

The methods and systems herein allow for confirming the identity of the sample, determining the purity of the sample, detecting contamination in the sample, determining an origin of contamination in the sample, and/or determining if the sample has sufficient genetic material for analysis.

In some embodiments, the nucleotide amplification method is PCR amplification. In some embodiments, the sequencing method is next-generation sequencing (NGS). In certain embodiments, the nucleotide amplification step comprises a step to add adapter sequences and barcodes for next-generation sequencing.

In certain embodiments, the method further comprises using an analysis system for calculating the frequency of the first allele and the second allele for each SNP and comparing the frequencies of the first allele and second alleles for each SNP to that of the reference sequence or the group of reference sequences in a sequence library.

In certain embodiments, the test sample is a sperm sample. The sperm sample may have been subjected to a machine for determining the sex of animal and comprises live and dead sperm. The sperm sample may have been subjected to UV irradiation.

In some embodiments, if the frequencies of the first alleles and second alleles for each SNP in the subset of SNPs are an exact match to those same SNPs in the reference sequence, then the test sample is at least 98% pure. In some embodiments, if the frequencies of the first alleles and second alleles for each SNP in the subset of SNPs are an exact match to those same SNPs in the reference sequence, then the test sample is at least 99% pure. In some embodiments, if the frequencies of the first alleles and second alleles for each SNP in the subset of SNPs are an exact match to those same SNPs in the reference sequence, then the test sample is at least 99.5% pure.

In some embodiments, if the test sample has 1 or more contaminating SNPs, the test sample is identified as contaminated. In some embodiments, if the test sample has 2 or more contaminating SNPs, the test sample is identified as contaminated. In some embodiments, if the test sample has 3 or more contaminating SNPs, the test sample is identified as contaminated.

The method may be performed as a multiplex assay, e.g., wherein at least 2 samples are subjected to the method simultaneously, wherein at least 48 samples are subjected to the method simultaneously, wherein at least 96 samples are subjected to the method simultaneously, etc.

In certain embodiments, the primer pool comprises at least 48 primer sets (e.g., 48 primer sets, 49 primer sets, 50 or more primer sets, etc.). In certain embodiments, the primer pool comprises 24 or more primer sets, 30 or more, 36 or more 40 or more, 42 or more, 45 or more, etc.

The methods may further comprise analyzing the sample to ensure the sample has adequate quantity. In certain embodiments, if the sample has fewer than 40 reads for each SNP in the primer pool (or particular subset of SNPs), then the sample has insufficient genetic material for analysis. In certain embodiments, if the sample has fewer than 35 reads for each SNP in the primer pool (or particular subset of SNPs), then the sample has insufficient genetic material for analysis. In certain embodiments, if the sample has fewer than 30 reads for each SNP in the primer pool (or particular subset of SNPs), then the sample has insufficient genetic material for analysis. In certain embodiments, if the sample has fewer than 25 reads for each SNP in the primer pool (or particular subset of SNPs), then the sample has insufficient genetic material for analysis. In certain embodiments, if the sample has fewer than 20 reads for each SNP in the primer pool (or particular subset of SNPs), then the sample has insufficient genetic material for analysis. In certain embodiments, if the sample has fewer than 45 reads for each SNP in the primer pool (or particular subset of SNPs), then the sample has insufficient genetic material for analysis. In certain embodiments, if the sample has fewer than 50 reads for each SNP in the primer pool (or particular subset of SNPs), then the sample has insufficient genetic material for analysis. If the sample does not have sufficient DNA for analysis, another DNA sample may be obtained to be retested.

In certain embodiments, the frequencies of non-matching alleles for the SNPs in the subset of SNPs is calculated as a number from 0-1.

In certain embodiments, the predetermined non-matching threshold is 0.5%. In certain embodiments, the predetermined non-matching threshold is 1%. In certain embodiments, the predetermined non-matching threshold is 2%. In certain embodiments, the predetermined non-matching threshold is 5%. In certain embodiments, the predetermined non-matching threshold is 0.5%, 1%, 2%, or 5%.

In certain embodiments, if the sample has at least 5 of the SNPs in the subset of SNPs that are contaminating SNPs, then the sample is considered a contaminated sample. In certain embodiments, if at least 5% of the SNPs in the subset of SNPs are contaminating SNPs, then the sample is considered a contaminated sample. In certain embodiments, wherein if at least 10% of the SNPs in the subset of SNPs are contaminating SNPs, then the sample is considered a contaminated sample. In certain embodiments, wherein if at least 15% of the SNPs in the subset of SNPs are contaminating SNPs, then the sample is considered a contaminated sample.

The method may further comprise determining an origin of contamination in the test sample. Determining the origin of contamination in the test sample may comprise comparing the test sample to one or more alternative reference sequences, wherein the contamination may be traced to the one or more alternative reference sequences. In certain embodiments, the alternative reference sequence is one from a sequence library, a public database, and/or an industry database.

In certain embodiments, the allele frequencies for each SNP are calculated by counting the number of reads that contain each allele on a 0-0.5 scale, wherein the smaller allele is used in the numerator; wherein genotypes are called on a 0,1,2 scale, wherein 0 is homozygous according to the reference sequence and 2 is homozygous but opposite the reference sequence, and 1 is heterozygous; wherein if the allele frequency is greater than or equal to 0.2 then the genotype is 1 or heterozygous, if the allele frequency is <2 and the allele is the same as the reference sequence then the genotype is 0 or homozygous or if it is opposite the reference sequence then the genotype is 2.

The present invention also provides a method of processing extracted DNA from a test sample. In certain embodiments, the method comprises subjecting the extracted DNA from the test sample to PCR amplification using a pool of SNP primer pairs, each SNP primer pair flanking a unique locus that contains a single SNP defining a first allele and a second allele, the nucleotide amplification produces amplicons for each SNP primer pair generating a pool of SNP amplicons; subjecting the pool of SNP amplicons to next-generation sequencing (NGS) to generate a nucleotide sequence for each amplicon in the pool, each amplicon being either the first allele or the second allele of a SNP; and calculating allele frequencies for the first allele and the second allele for each SNP and comparing the allele frequencies for the first alleles and second alleles for each SNP in a subset of SNPs to those same SNPs in a reference sequence, the subset of SNPs is a group of SNPs expected to be homozygous. In certain embodiments, allele frequencies for each SNP are calculated by counting the number of reads that contain each allele on a 0-0.5 scale, wherein the smaller allele is used in the numerator; wherein genotypes are called on a 0,1,2 scale, wherein 0 is homozygous according to the reference sequence and 2 is homozygous but opposite the reference sequence, and 1 is heterozygous; wherein if the allele frequency is greater than or equal to 0.2 then the genotype is 1 or heterozygous, if the allele frequency is <2 and the allele is the same as the reference sequence then the genotype is 0 or homozygous or if it is opposite the reference sequence then the genotype is 2. In certain embodiments, if the frequencies of the first alleles and second alleles for each SNP in the subset of SNPs are an exact match to those same SNPs in the reference sequence, then the test sample is the same as that of the reference sequence and the sample is at least 95% pure. In certain embodiments, if the frequencies of the first alleles and second alleles for each SNP in the subset of SNPs are not exact matches, then a frequency of non-matching alleles is calculated, wherein if the frequency of the non-matching alleles is above a predetermined non-matching threshold then the SNP is a contaminating SNP, wherein the method further comprises calculating a number of contaminating SNPs in the test sample, wherein if the number of contaminating SNPs is above a predetermined contaminating SNP threshold, then the sample is identified as having a contamination.

As previously discussed, the methods and systems herein allow for confirming the identity of the sample, determining the purity of the sample, detecting contamination in the sample, determining an origin of contamination in the sample, and/or determining if the sample has sufficient genetic material for analysis. As such, the present invention provides a method of confirming the identity of a sample; a method of determining the purity of a sample; a method of detecting contamination in a sample; a method of determining the origin of contamination in the sample; and a method of determining if the sample has sufficient genetic material for analysis. These methods incorporate the aforementioned steps in the method of processing samples, e.g., subjecting the extracted DNA from the test sample to nucleotide amplification using a pool of SNP primer pairs (wherein each SNP primer pair flanks a unique locus that contains a single target SNP defining a first allele and a second allele, wherein the nucleotide amplification produces amplicons for each SNP primer pair generating a pool of SNP amplicons); subjecting the pool of SNP amplicons to sequencing to generate a nucleotide sequence for each amplicon in the pool; calculating a frequency of the first allele and the second allele for each SNP, etc.

As an example, the present invention provides a method of processing extracted DNA from a test sample for detecting a genetic match. In certain embodiments, the method comprises subjecting the extracted DNA from the test sample to nucleotide amplification using a pool of SNP primer pairs, each SNP primer pair flanking a unique locus that contains a single target SNP defining a first allele and a second allele, the nucleotide amplification produces amplicons for each SNP primer pair generating a pool of SNP amplicons; subjecting the pool of SNP amplicons to sequencing to generate a nucleotide sequence for each amplicon in the pool; and calculating a frequency of the first allele and the second allele for each SNP. The frequencies of the first alleles and second alleles for each SNP may be compared. For example, in certain embodiments, the frequencies of the first alleles and the second alleles for each SNP in a subset of the target SNPs to a reference sequence, wherein the subset of target SNPs is a group of SNPs expected to be homozygous. If the frequencies of the first alleles and second alleles for each SNP in the subset of target SNPs are an exact match to the corresponding SNPs in the reference sequence, then the test sample is the same as that of the reference sequence, e.g., there is a genetic match.

As another example, the present invention provides a method of processing extracted DNA from a test sample to detect possible contamination. In certain embodiments, the method comprises subjecting the extracted DNA from the test sample to nucleotide amplification using a pool of SNP primer pairs, each SNP primer pair flanking a unique locus that contains a single target SNP defining a first allele and a second allele, the nucleotide amplification produces amplicons for each SNP primer pair generating a pool of SNP amplicons; subjecting the pool of SNP amplicons to sequencing to generate a nucleotide sequence for each amplicon in the pool; and calculating a frequency of the first allele and the second allele for each SNP. The frequencies of the first alleles and second alleles for each SNP may be compared. For example, in certain embodiments, the frequencies of the first alleles and the second alleles for each SNP in a subset of the target SNPs to a reference sequence, wherein the subset of target SNPs is a group of SNPs expected to be homozygous. If the frequencies of the first alleles and second alleles for each SNP in the subset of SNPs are not exact matches to the corresponding SNPs in the reference sequence, then a frequency of non-matching alleles is calculated for the SNPs in the subset of SNPs (e.g., the SNPs of the subset that are not exact matches). If the frequency of the non-matching alleles for a particular SNP is above a predetermined threshold (e.g., a “non-matching threshold”), then the particular SNP is considered to be a contaminating SNP (e.g., a potential indication of a mixture or error), and if the number of contaminating SNPs is above a predetermined threshold (e.g., a “contaminating SNP threshold”), then the sample is identified as having a potential contamination (e.g., the sample is considered to be a potential mixture or contain errors).

The present invention also provides systems, e.g., a computer-implemented system, an analysis system, for performing the methods disclosed herein. For example, the systems may feature a processor (e.g., microprocessor) for performing mathematical and/or statistical and/or analytical operations for one or more steps in the methods disclosed herein. The processor (e.g., microprocessor) may be operatively connected to one or more other components of the system, e.g., a sequencing system, a user interface, etc.

The methods herein may be computer-implemented methods.

With respect to the methods disclosed herein, the methods may further comprise additional steps related to the sale and/or use of the samples from which the test samples are derived, e.g., the semen straws from which the test samples were derived. In certain embodiments, the methods further comprise “approving” or “passing” a sample wherein there is a genetic match, e.g., labeling the sample in some way to indicate that the sample may be used and/or sold. In certain embodiments, the methods further comprise providing an approved (passed) sample for sale and/or use. In certain embodiments, the methods further comprise retesting the sample if it is considered to be possibly contaminated. In certain embodiments, the methods further comprise “failing” a sample wherein there is a contaminant or error, e.g., labeling the sample in some way to indicate that the sample may not be used and/or sold. In certain embodiments, the methods further comprise destroying the sample from which the test sample is derived if the sample is determined to be contaminated.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows the results from a series of deliberate mixtures.

FIG. 1B shows the frequency of the second allele for a single-nucleotide polymorphism (SNP) expected to be homozygous.

FIG. 1C shows the evidence of mixing compared to the background noise for various samples with particular fractions of contamination from a second bull.

FIG. 2 shows an example of the second allele frequencies for particular SNPs expected to be homozygous in a real sample with a contaminating bull. There are three categories of SNPs: “0” represents 0 copies of the second allele; the dark grey boxes (with numbers 37, 30, 34, and 35) represent 2 copies of the second allele, and the remaining numbered boxes (with numbers 17, 17, 13, 19, 20, 19, and 14) represent one copy of the second allele. This serves as a fingerprint for identifying the contaminating bull. For example, bull 29HO17718 is the only bull in the group with that particular genotype.

FIG. 3A shows a schematic of a workflow related to the methods of the present invention. The present invention is not limited to the workflow in FIG. 3A. Note that samples that are non-100% matches may be subject to retesting before being destroyed.

FIG. 3B shows a schematic of a workflow related to the methods of the present invention. The present invention is not limited to the workflow in FIG. 3B.

FIG. 4 shows a schematic of a workflow related to the methods of the present invention, wherein the sequencing data is stored in a database that is integrated or operatively connected to an analysis system with a user interface. The user interface allows for review and quality control of the samples.

FIG. 5 shows a non-limiting example of output data for a group of hypothetical straw samples.

### DETAILED DESCRIPTION OF THE INVENTION

The present invention features methods and systems for processing genetic samples and testing the purity of samples (e.g., semen straws), e.g., for detecting a suspected mixture or contamination, for detecting the mislabeling of a sample (e.g., sample swap), for confirming the identity of a sample, for determining the identity (origin) of the sample, for determining the identity (origin) of contamination, if any, etc.

The methods for processing the genetic samples herein feature amplifying particular regions of extracted DNA from test samples (e.g., sperm sample, e.g., sperm samples derived from semen straws), sequencing the amplicons, and analyzing the amplicons.

Samples may be provided to a user as extracted DNA. Alternatively, one of ordinary skill in the art understands that in the case of a raw sample, an additional step of DNA extraction may be performed before further processing.

### Amplification and Sequencing of Amplicons

Referring to the methods and systems herein, the genetic samples (e.g., extracted DNA) are subjected to amplification (e.g., PCR amplification), wherein the amplification step produces amplicons of specific SNPs. Methods of DNA amplification are well known to one of ordinary skill in the art. For example, amplification may refer to but is not limited to PCR amplification. Sets of SNP primer pairs, wherein each SNP primer pair flank a unique locus (a target SNP), thus defining a first allele and a second allele, are used as primers during amplification.

In certain embodiments, the primer pool comprises at least 24 primer sets (for amplification of 24 target SNPs). In some embodiments, the primer pool comprises at least 36 primer sets (for amplification of 36 target SNPs). In some embodiments, the primer pool comprises at least 40 primer sets (for amplification of 40 target SNPs). Iln some embodiments, the primer pool comprises at least 48 primer sets (for amplification of 48 target SNPs). In some embodiments, the primer pool comprises 48 primer sets. In some embodiments, the primer pool comprises 49 primer sets. In some embodiments, the primer pool comprises 50 primer sets. In some embodiments, the primer pool comprises 24 to 48 primer sets. In some embodiments, the primer pool comprises 40 to 50 primer sets. In some embodiments, the primer pool comprises 48 or 49 primer sets. In some embodiments, the primer pool comprises 50 to 60 primer sets. In some embodiments, the primer pool comprises more than 50 primer sets.

In certain embodiments, at least 10 of the target SNPs are expected to be homozygous. In certain embodiments, at least 15 of the target SNPs are expected to be homozygous. In certain embodiments, at least 20 of the target SNPs are expected to be homozygous. In certain embodiments, at least 25 of the target SNPs are expected to be homozygous. In certain embodiments, at least 30 of the target SNPs are expected to be homozygous. In certain embodiments, at least 35 of the target SNPs are expected to be homozygous. In certain embodiments, at least 10% of the target SNPs are expected to be homozygous. In certain embodiments, at least 20% of the target SNPs are expected to be homozygous. In certain embodiments, at least 25% of the target SNPs are expected to be homozygous. In certain embodiments, at least 30% of the target SNPs are expected to be homozygous. In certain embodiments, at least 40% of the target SNPs are expected to be homozygous. In certain embodiments, at least 50% of the target SNPs are expected to be homozygous. In certain embodiments, at least 60% of the target SNPs are expected to be homozygous. In certain embodiments, at least 70% of the target SNPs are expected to be homozygous. In certain embodiments, at least 75% of the target SNPs are expected to be homozygous. In certain embodiments, all of the target SNPs are expected to be homozygous.

Examples of primer pairs used in the methods of the present invention are described below in Table 1. It is to be understood that the primers disclosed herein are provided as an example only, and the present invention is not limited to the primers nor the SNPs disclosed herein. Based on the disclosure of the present invention, one of ordinary skill in the art would be capable of selecting other SNPs and designing primers for said selected SNPs. Likewise, the present invention is not limited to testing of sperm samples in bulls; the methods and systems herein may be applied to other sample types and other species (e.g., other mammals).

TABLE 1

| Primer | SEQ ID | | |
|---|---|---|---|
| pair | NO: | Sequence | Chromosome |
| 1 | 1 | TTTCCTTCTGTAGATGTTAACTGGT | 5 |
| | 2 | ACGGACAATAAACTGTAAATTTC | |
| 2 | 3 | CACTTGTATGTATTTCAGAAGTTTTC | 25 |
| | 4 | AGGATCACAAACAATGCCCT | |
| 3 | 5 | TCTGAAGGAATTGAAAATGTCTACCA | 20 |
| | 6 | ACTTCTACAATTAGCGATTAACTG | |
| 4 | 7 | TCAGAGGAGAATGTCTAGTTTAGA | 14 |
| | 8 | TCAGTGTGACGGAGCTGC | |
| 5 | 9 | TCATGAGAAATCAGCCCACA | 27 |
| | 10 | ACCTGCTGTGTAATGTATTTAAAC | |
| 6 | 11 | AGGTTTAAGGGAATATTTGCACCT | 19 |
| | 12 | TTTGAATAACTGTACAGGGAATTC | |
| 7 | 13 | GCTTAAAGTTCTAAACCAATCAACA | 1 |
| | 14 | AGCTCTTCTAAAACAAGTAAAGCCA | |
| 8 | 15 | CCAAGAACCACTGTGATAGGAG | 22 |
| | 16 | TAAGAAAAGAGGGAACAAGACT | |
| 9 | 17 | ATGTGGCTTCCTGTATTCCCT | 13 |
| | 18 | GTTCAGCAAATAATTTATAGAGAACC | |
| 10 | 19 | CTGGCCCAAACTCATCACAC | 8 |
| | 20 | ACAAACCAACCACCAAACAGA | |
| 11 | 21 | TGAGTTTCAGAGAGGGCCAG | 3 |
| | 22 | GCCACTTGATGCCATAGGTC | |
| 12 | 23 | AGAAAGCCATACCCAGGGAG | 11 |
| | 24 | AGACAACAGTGAAGTTCAGGC | |
| 13 | 25 | CCCATGTATGTGTAGCTGGC | 4 |
| | 26 | CTGAGGAAGACAGGGAGAAGG | |
| 14 | 27 | ACATAAGTACATATCTACTGGCCT | 2 |
| | 28 | TGAGAACCACTTAAGATAGGGT | |
| 15 | 29 | TACTAATTGTATATCTTGCTGCTCAG | 3 |
| | 30 | CTGCCTTCCCTCCTCAGTC | |
| 16 | 31 | AGGTATTATGAGTTGTGTGGGT | 4 |
| | 32 | ACATCCTCTTACCTAATCTGAGCT | |
| 17 | 33 | GGAAACCCTATGAGCCAGAGT | 6 |
| | 34 | TCCCTCAGCCCCTTTCAG | |
| 18 | 35 | GGGAGTGGAGAGTGGATTGG | 13 |
| | 36 | TGCCCCTTTTGTACAGATGG | |
| 19 | 37 | ATTCTCTTGACTTGCAGGCG | 29 |
| | 38 | GTCAATAACTCAATAAAAGCACTGA | |
| 20 | 39 | TGGGTTCTTGGGTCCAGAG | 18 |
| | 40 | AGGTCCCAGTCTCCTCCTTC | |
| 21 | 41 | GACTGCCTGGATCTGAGAGG | 19 |
| | 42 | CAACGGGACACTTGCCTTTC | |
| 22 | 43 | ATGTCTAGCCCTCACTCCCA | 24 |
| | 44 | CGTGATCAAGGCAGAACTACA | |
| 23 | 45 | AAGAGCAGCTGGTTTCCTAT | 2 |
| | 46 | AGGGGAGAAGATATGCAGACAG | |
| 24 | 47 | GTCCTCATCAGCCTCGTCAT | 2 |
| | 48 | ATATCCACCCTGCAAACCCA | |
| 25 | 49 | AGTGCTATCATGTGCCTTGA | 3 |
| | 50 | ACACAGACTTTAAAGCAGCCT | |
| 26 | 51 | GAGGGGAGGGGAGGGTTG | 5 |
| | 52 | GAGAATTCAGGGGCCCTTCT | |
| 27 | 53 | GGATAATCTTTAGCAATCAGAGGC | 7 |
| | 54 | TCTCCCCGCTTGTAACAGTT | |
| 28 | 55 | AAAACTTGCTGCCAGGGAA | 2 |
| | 56 | CACGAGGCACATATCAGT | |
| 29 | 57 | CCTGTTACCTGGGCTTCATG | 10 |
| | 58 | ACGACTCGGCATAGATGATTTG | |
| 30 | 59 | CCTCTCTTTCAGGCCAAAATCC | 8 |
| | 60 | CTATTCTATTTTCCTCAAGTATCTGC | |
| 31 | 61 | ATTCAGATTGATGGTCCAGCA | 15 |
| | 62 | AGTGGTGATCTCAAAGAGGCT | |
| 32 | 63 | TAACTGCATCGTTAAACTGGCT | 20 |
| | 64 | CTTCTGGAGCTCACCCACC | |
| 33 | 65 | CACTCAGACGTCCCCAACC | 10 |
| | 66 | GGGTTCGGCAAGATTCAAGT | |
| 34 | 67 | GAGGCTGCCAGGTGTCAC | 24 |
| | 68 | TGAAAAGGTAGCCCAGGTGT | |
| 35 | 69 | GTTTTGCCTTCCACCCACC | 13 |
| | 70 | TCTCCAAAGCAAACAAGTTAGTG | |
| 36 | 71 | CCAACCTGTTCAGCACA | 26 |
| | 72 | AGATCTTCCCCAACAGTACT | |
| 37 | 73 | GGAAGTCCCTTGTGCTCATC | 21 |
| | 74 | AAGTAGACCACGCCCCTTTC | |
| 38 | 75 | AAAGTAATAAGGCTTGCCTTCCA | 2 |
| | 76 | TGAGAAATATTGACTACACGCCC | |
| 39 | 77 | AAGGGGCTCATAAGATAAAGC | 1 |
| | 78 | CCAGAGATGGGGCATAGAACT | |
| 40 | 79 | GTGCCTGGCTCTGTACA | 3 |
| | 80 | TGCCGACCTCACGTGG | |
| 41 | 81 | GTCCATCCATGTTGCTGCAA | 10 |
| | 82 | TACTGGCCAAGAACACCT | |
| 42 | 83 | ATTATGTTTTAATGCACTGCTGT | 11 |
| | 84 | TTGGCATTGGGAAAGGGAAA | |
| 43 | 85 | TGTGGGTTGTAGTAGCAGCA | 14 |
| | 86 | ACCCAGTCACATTCAGAGCT | |
| 44 | 87 | ACTAAAATACAAATGCAGGCAC | 10 |
| | 88 | GGTACCCTGTATTCTTTCAAATGAGT | |
| 45 | 89 | TGCTGCAGATTCTTTACTTGCT | 21 |
| | 90 | TCTCTAGCCCATGTCATGACT | |
| 46 | 91 | TCTTTTCATCCTTACCATAGCTAGG | 28 |
| | 92 | TTGCAGATGTGATCAAGGCT | |
| 47 | 93 | AGGATTGTATTTAAGGTTGTCTGA | 18 |
| | 94 | TGATGCCCACAATACCAGGT | |
| 48 | 95 | CAGGCAGATGGTTTTAACACAC | 28 |
| | 96 | AGATAATATAGACTGTAGTGCTGGGT | |

The primer pairs allow for amplification of specific SNPs, wherein at least a subset of the SNPs can help determine the identity and purity of the test sample.

The pool of SNP amplicons resulting from the amplification step are further subjected to sequencing. Methods of sequencing, such as but not limited to next-generation sequencing (NGS), are well known to one of ordinary skill in the art. Additional steps in the method may include attachment of adapter sequences for NGS.

### Analysis

The results from the sequencing are provided to an analysis system, e.g., an application, e.g., a computer-based system for compiling and/or organizing and/or performing mathematical or statistical operations on the information obtained from sequencing. For example, the analysis system may calculate the frequency of the first allele and the second allele for each SNP. As is described herein, the analysis system compares the sequences and/or allele frequencies for each SNP to the corresponding SNPs of at least one reference sequence (e.g., a library sequence, a sequence from a known individual, e.g., a known bull, etc.). A subset of the corresponding SNPs of the reference sequence (e.g., library sequence, sequence from a reference individual, e.g., known bull, etc.) are homozygous SNPs. In certain embodiments, the method comprises calculating the frequency of a non-matching allele for each SNP in the test sample corresponding to each expected homozygous SNP in the reference sequence. In some embodiments, particular calculations are based on the frequency of a non-matching allele for each expected homozygous SNP and the number of SNPs with a particular frequency of non-matching alleles, allowing for the determination of a rate of contamination by one or more genetically distinct individuals. The analysis helps to detect a genetic match (e.g., confirm identity), to determine the identity of the genetic sample, detect a potential mixture, and/or determine origin of contaminating material, etc.

In some embodiments, the methods and systems feature a step that checks the quantity of the DNA in the sample to determine if there is enough genetic material amplified and sequenced so as to be properly analyzed. If there is not enough DNA for proper analysis, the system may produce an error signal, e.g., “LOW” signal. In certain embodiments, DNA extraction is repeated.

With respect to the quantity of the sample, the analysis system may calculate the frequencies of all target SNPs or a particular subset of SNPs (e.g., the subset of SNPs expected to be homozygous, etc.). The methods and systems may require a certain number of reads for each of the SNPs in the predetermined subset of SNPs (or all target SNPs if the system uses all for quantity analysis). For example, in certain embodiments, the methods and systems require at least 40 reads of each SNP (or a subset of the SNPs). In certain embodiments, at least 35 reads of each SNP (or a subset of the SNPs). In certain embodiments, at least 30 reads of each SNP (or a subset of the SNPs). In certain embodiments, at least 25 reads of each SNP (or a subset of the SNPs). In certain embodiments, at least 20 reads of each SNP (or a subset of the SNPs). In certain embodiments, at least 45 reads of each SNP (or a subset of the SNPs). In certain embodiments, at least 50 reads of each SNP (or a subset of the SNPs). The present invention is not limited to the aforementioned SNP requirements.

In certain embodiments, the subset of SNPs used for the quantity analysis comprises 5 or more SNPs. In certain embodiments, the subset of SNPs used for the quantity analysis comprises 10 or more SNPs. In certain embodiments, the subset of SNPs used for the quantity analysis comprises 15 or more SNPs. In certain embodiments, the subset of SNPs used for the quantity analysis comprises 20 or more SNPs. In certain embodiments, the subset of SNPs used for the quantity analysis comprises 25 or more SNPs. In certain embodiments, the subset of SNPs used for the quantity analysis comprises 30 or more SNPs. In certain embodiments, the subset of SNPs used for the quantity analysis comprises 35 or more SNPs. In certain embodiments, the subset of SNPs used for the quantity analysis comprises 40 or more SNPs. In certain embodiments, the subset of SNPs used for the quantity analysis comprises all of the target SNPs. In certain embodiments, the subset of SNPs used for the quantity analysis is all of the SNPs expected to be homozygous.

The overall composition of SNPs and their ratios, relative to a defined standard (e.g., sequence library, reference sequences, etc.), allows for testing for identity and an estimation of a degree of contamination by one or more genetically distinct individuals, if applicable.

The analysis system is configured to calculate the frequencies of the alleles for each SNP (or at least a subset of SNPs such as a subset of SNPs expected to be homozygous). For example, the analysis system is configured to calculate the frequency of the second allele (non-matching allele) for the target SNPs expected to be homozygous. The purity analysis portion of the methods herein relies on a reference genotype and focuses on the SNPs that should be homozygous in the expected genotype. If the SNP should be homozygous, detection of the other allele (second allele, non-matching allele) for that SNP in the sample would be unexpected (or at least detection of a large amount of the other allele would be unexpected, since a certain level of noise is typical with sequencing). Thus, the frequency of the other allele should be below a particular predetermined threshold (e.g., a non-matching allele frequency threshold, as described below).

Allele frequencies may be represented as a percentage, as a number from 0-1, etc. For example, if there are no second alleles detected for a particular expected-homozygous SNP, the allele frequency may be represented as 0. Detection of the second allele may result in allele frequencies greater than 0 (and up to 1), if using a scale from 0-1, such as 0.03, 0.05, 0.1, 0.18, 0.3, etc.

As previously discussed, the analysis system may use a second allele (or non-matching allele) frequency threshold, wherein detection of a non-matching allele frequency at and/or above the predetermined threshold indicates the SNP is a “contaminating” SNP. In certain embodiments, a non-matching allele frequency greater than 0 is indicative of a contaminating SNP. In other words, in some embodiments, the non-matching allele frequency threshold may be anything greater than 0. In certain embodiments, the non-matching allele frequency threshold (e.g., the frequency of the second allele that would indicate that the SNP is a contaminating SNP) is 0.005, or 0.5%. In certain embodiments, the non-matching allele frequency threshold (e.g., the frequency of the second allele that would indicate that the SNP is a contaminating SNP) is 0.01, or 1%. In certain embodiments, the non-matching allele frequency threshold (e.g., the frequency of the second allele that would indicate that the SNP is a contaminating SNP) is 0.02, or 2%. In certain embodiments, the non-matching allele frequency threshold (e.g., the frequency of the second allele that would indicate that the SNP is a contaminating SNP) is 0.03, or 3%. In certain embodiments, the non-matching allele frequency threshold (e.g., the frequency of the second allele that would indicate that the SNP is a contaminating SNP) is 0.04, or 4%. In certain embodiments, the non-matching allele frequency threshold (e.g., the frequency of the second allele that would indicate that the SNP is a contaminating SNP) is 0.05, or 5%.

The purity check generally uses the homozygote called genotype from a sample to classify the sample as having a mixture. For example, in some embodiments, clean homozygotes are expected to have a (maf<0.02), and clean heterozygotes are expected to have a (maf>0.4). A mixture may be a SNP called homozygote with a (0.02<maf<0.4).

The detection of a certain number of contaminating SNPs above a predetermined threshold is indicative of a mixture or possible mixture (e.g., genetic contamination) in the sample. For example, detection of 1 or more contaminating SNPs is indicative of a mixture (e.g., genetic contamination) in the sample. In some embodiments, 2 or more contaminating SNPs is indicative of a mixture (e.g., genetic contamination) in the sample. In some embodiments, 3 or more contaminating SNPs is indicative of a mixture (e.g., genetic contamination) in the sample. In some embodiments, 4 or more contaminating SNPs is indicative of a mixture (e.g., genetic contamination) in the sample. In some embodiments, 5 or more contaminating SNPs is indicative of a mixture (e.g., genetic contamination) in the sample. In some embodiments, 6 or more contaminating SNPs is indicative of a mixture (e.g., genetic contamination) in the sample. In some embodiments, 7 or more contaminating SNPs is indicative of a mixture (e.g., genetic contamination) in the sample. In certain embodiments, a sample wherein at least 1% of the expected-homozygous SNPs are marked as contaminating SNPs is considered a mixed or contaminated sample. In certain embodiments, a sample wherein at least 2% of the expected-homozygous SNPs are marked as contaminating SNPs is considered a mixed or contaminated sample. In certain embodiments, a sample wherein at least 3% of the expected-homozygous SNPs are marked as contaminating SNPs is considered a mixed or contaminated sample. In certain embodiments, a sample wherein at least 4% of the expected-homozygous SNPs are marked as contaminating SNPs is considered a mixed or contaminated sample. In certain embodiments, a sample wherein at least 5% of the expected-homozygous SNPs are marked as contaminating SNPs is considered a mixed or contaminated sample. In certain embodiments, a sample wherein at least 10% of the expected-homozygous SNPs are marked as contaminating SNPs is considered a mixed or contaminated sample. In certain embodiments, a sample wherein at least 15% of the expected-homozygous SNPs are marked as contaminating SNPs is considered a mixed or contaminated sample. In certain embodiments, a sample wherein at least 20% of the expected-homozygous SNPs are marked as contaminating SNPs is considered a mixed or contaminated sample. In certain embodiments, a sample wherein at least 25% of the expected-homozygous SNPs are marked as contaminating SNPs is considered a mixed or contaminated sample.

In certain embodiments, purity (or amount of contamination) is reported by taking the median of the 3 highest frequency SNPs. In certain embodiments, purity (or amount of contamination) is reported by taking the median of the 4 highest frequency SNPs. In certain embodiments, purity (or amount of contamination) is reported by taking the median of the 3 highest frequency SNPs. In certain embodiments, purity (or amount of contamination) is reported by taking the median of the 5 highest frequency SNPs. The present invention is not limited to the aforementioned method of reporting purity or amount of contamination.

In certain embodiments, a contaminated sample is one that has a median frequency of the highest three contaminating SNPs greater than 5%. In certain embodiments, a contaminated sample may be one that has a median frequency of the highest three contaminating SNPs greater than 10%. In certain embodiments, a contaminated sample may be one that has a median frequency of the highest three contaminating SNPs greater than 15%. In certain embodiments, a contaminated sample may be one that has a median frequency of the highest three contaminating SNPs greater than 20%. In certain embodiments, a contaminated sample may be one that has a median frequency of the highest three contaminating SNPs greater than 25%.

In some embodiments, the amount of contamination in the sample is 1% or less. In some embodiments, the amount of contamination in the sample is 2% or less. In some embodiments, the amount of contamination in the sample is 3% or less. In some embodiments, the amount of contamination in the sample is 4% or less. In some embodiments, the amount of contamination in the sample is 5% or less. In some embodiments, the amount of contamination in the sample is 1-2% or less. In some embodiments, the amount of contamination in the sample is 2-3% or less. In some embodiments, the amount of contamination in the sample is 2-4% or less. In some embodiments, the amount of contamination in the sample is 2-5% or less. In some embodiments, the amount of contamination in the sample is 5-10% or less. In some embodiments, the amount of contamination in the sample is 2-15% or less. In some embodiments, the amount of contamination in the sample is 1% or more. In some embodiments, the amount of contamination in the sample is 2% or more. In some embodiments, the amount of contamination in the sample is 3% or more. In some embodiments, the amount of contamination in the sample is 5% or more.

As a non-limiting example, in certain embodiments, at least 20% of the expected-homozygous SNPs must have a level of contamination of at least 1% to cause the sample to be flagged as contaminated. In certain embodiments, at least 10% of the expected-homozygous SNPs must have a level of contamination of at least 1% to cause the sample to be flagged as contaminated. In certain embodiments, at least 20% of the expected-homozygous SNPs must have a level of contamination of at least 2% to cause the sample to be flagged as contaminated. In certain embodiments, at least 10% of the expected-homozygous SNPs must have a level of contamination of at least 2% to cause the sample to be flagged as contaminated. In certain embodiments, at least 20% of the expected-homozygous SNPs must have a level of contamination of at least 3% to cause the sample to be flagged as contaminated. In certain embodiments, at least 10% of the expected-homozygous SNPs must have a level of contamination of at least 3% to cause the sample to be flagged as contaminated.

As previously discussed, calculation of the second allele frequencies (or non-matching allele frequencies) allows the sample to be tested for identity, to confirm whether the semen is from the correct animal, to determine the animal from which it originated, to identify a sample swap, to identify the origin of contamination, etc.

With respect to testing for identity, the genotype in the sample may be determined by checking against reference sequences, such as but not limited to the Council on Dairy Cattle Breeding (CDCB) database. If the sample has the expected genotype (e.g., if all of the expected-homozygous SNPs match those of the expected genotype), the sample may pass. If not, the sample may be checked against a previously sequenced genotype (a stand-in reference) such as but not limited to previously sequenced samples in the laboratory (previous QC samples), other industry samples, historical samples, etc. (e.g., a public database, though some animals may not be in a public database). If the sample is the reference sequence used as the stand-in reference, the sample may pass. If the sample does not pass, it may be subjected to further analysis and/or testing.

The methods and systems may be able to identify the combinations of individuals in a mixed test sample based on the sequences in a sequence library (e.g., animal #1 plus animal #2, animal #1 plus animal #2 plus animal #3, etc.). In certain embodiments, the sample may be determined to be a sample swap or a mislabeling.

The present invention is not limited to the aforementioned parameters or combinations of parameters that are used to calculate the level of contamination in the sample. Generally, the overall composition of SNPs and their ratios, relative to the defined standard, allows an estimation of the degree of contamination by one, or more, genetically distinct individuals. Final results may take on the form of number of mismatches, whether or not the sample is a sample swap, a percentage of mixture, a percentage of contamination, a list of potential contaminants, etc. Other results may include the allele frequencies for each SNP. As previously discussed, identification of a contaminated sample or mixture can result in the re-testing of a sample. The sample may then be determined to be pure. Inaccuracies with respect to the purity (e.g., pure sample, mixed sample) may be due to a variety of circumstances such as but not limited to errors in the sequencing process, contamination by a technician during sample preparation, etc.

In certain embodiments, the identity of the contaminant is determined. For example, a search may be run against reference genotypes, e.g., reference genotypes of samples with high likelihood of being the contaminant. In some embodiments, a contaminant may be identified by comparing the results to all the sequences within that day's sequencing run.

As previously discussed, the DNA sequences of the SNPs in the sample may be compared to one or more reference sequences. In certain embodiments, the reference sequence is a sequence in a sequence library. In certain embodiments, the reference sequence is one or more sequences of a group of bulls, e.g., a bull matching the sperm sample being tested, bulls not matching the sperm sample being tested, bulls that may be responsible for contamination of the sperm sample in the straw, bulls in a particular group or cohort, etc. For example, a reference library may comprise the sequences (each with its own unique SNP profile) from a group of bulls. As previously discussed, comparisons with reference sequences may help to confirm the identity of the sample, identify which bull the sperm sample belongs to, and/or which bull is the origin of the contamination, if any.

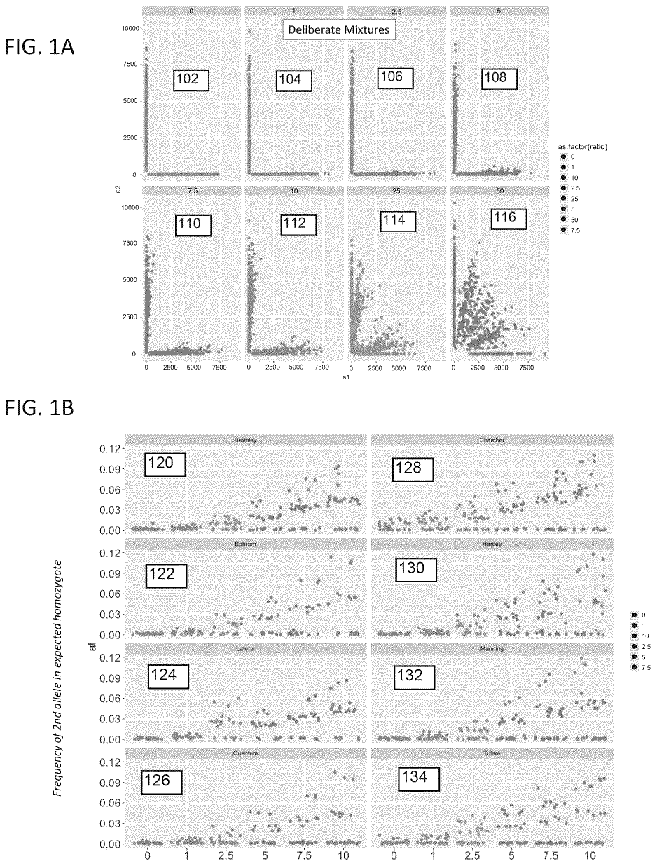

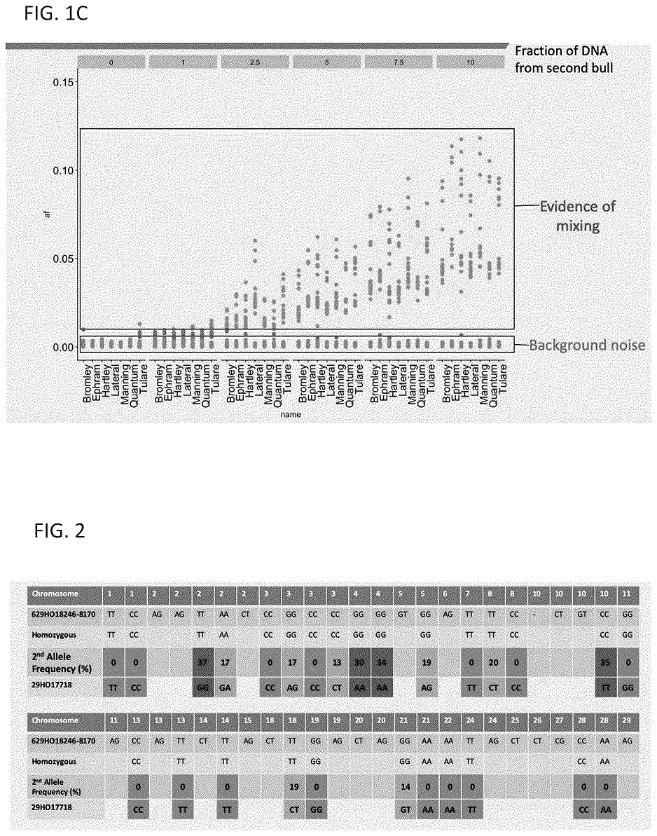

FIG. 1A shows a series of deliberate mixtures illustrating the results for mixed or contaminated samples. Chart 102 shows a mixture ratio of 0. Chart 104 shows a mixture ratio of 1. Chart 106 shows a mixture ratio of 2.5. Chart 108 shows a mixture ratio of 5. Chart 110 shows a mixture ratio of 7.5. Chart 112 shows a mixture ratio of 10. Chart 114 shows a mixture ratio of 25. Chart 116 shows a mixture ratio of 50. In FIG. 1B, charts 120, 122, 124, 126, 128, 130, 132, and 134 shows the frequency of the second allele for a SNP expected to be homozygous at mixing ratios of 0, 1, 2.5, 2, 5, 7.5, and 10 for respective individuals Bromley, Ephram, Lateral, Quantum, Chamber, Hartley, Manning, and Tulare. FIG. 1C shows the evidence of mixing compared to the background noise for various samples with particular fractions of contamination from a second bull.

FIG. 2 shows an example of second allele frequencies for particular SNPs expected to be homozygous. In the exemplary chart in FIG. 2, the SNPs at chromosomes 2, 4, and 10 have second allele frequencies occurring at or greater than 30%, and the chromosomes 2, 3, 5, 8, 18, and 21 have second allele frequencies occurring above 0% but below 30%. This serves as a fingerprint for identifying the contaminating bull. For example, bull 29HO17718 is the only bull in the group with that particular genotype.

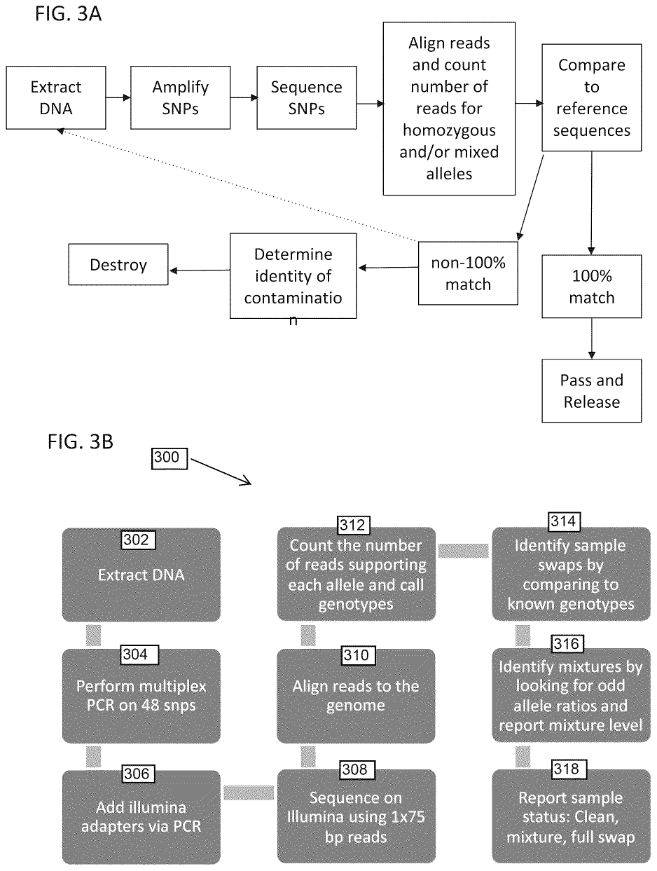

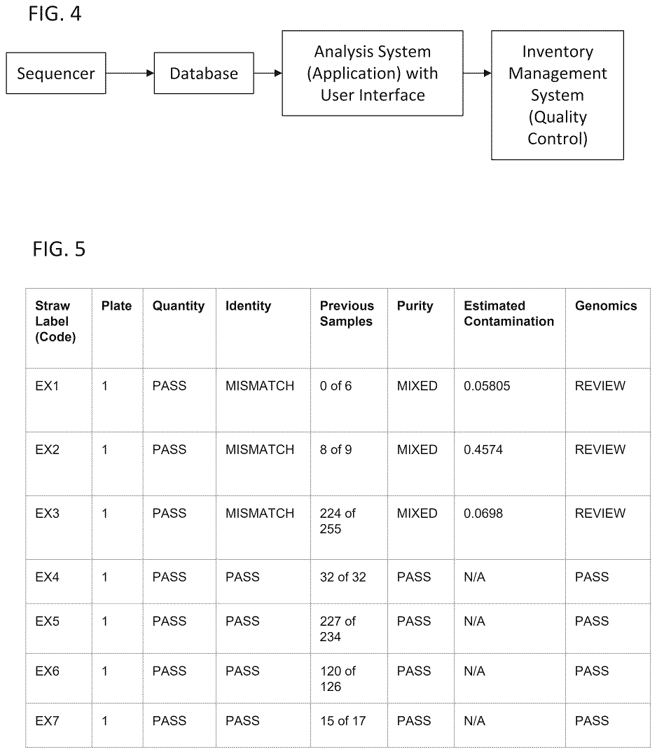

FIG. 3A, FIG. 3B, and FIG. 4 show schematic representations of the methods and systems and workflows of the present invention. Note that samples that are non-100% matches (or labeled as mixed or possibly mixed) may be subject to retesting before being destroyed.

### Example 1

The following is a non-limiting example of the present invention. It is to be understood that said example is not intended to limit the present invention in any way. Equivalents or substitutes are within the scope of the present invention.

First, DNA extraction is performed using standard methods. A multiplex PCR is then performed using Qiagen Multiplex PCR Master Mix (Cat no: 206145) and 48 combined primer pairs. The product is then bead cleaned and verified for amplification on a gel. A second PCR is run to add the Illumina adapter sequences and barcodes. The samples are then pooled and sequenced on a next-generation sequencer (NGS) such as an Illumina sequencer using 1×75 bp reads. The samples are demultiplexed and binned by barcode by bcl2fastq. The fastqs are aligned to the UMD3.1 genome using BWA MEM. Allele frequencies (AF) for each SNP are calculated by counting the number of reads that contain each allele on a 0-0.5 scale, where the smaller allele is used in the numerator. Genotypes (GT) are called on a 0,1,2 scale where 0 is homozygous reference and 2 is homozygous alternate. 1 is heterozygous. If the AF is greater than or equal to 0.2 the GT is 1 (heterozygous). If the AF is <2 and the observed allele is reference the GT is 0, otherwise it is GT is 2. Once the genotypes are called, the identity of the sample can be checked by comparing the observed genotype with the genotypes in the database. If the sample does not match the genotypes in the database with less than 4 mismatches, then the Council of Dairy Cattle Breeding database (CDCB) is scanned for perfect matches. If a hit with >95% match is found the top hit is reported and the sample is labeled a Full swap. To detect contamination, SNPs that are expected to be homozygous are first selected. If a contamination occurs, these SNPs are expected to have an AF >0.02. By checking for multiple SNPs for these unexpected AF it can be determined if a sample has contamination or not. Using the top 3 SNPs, contamination level can be estimated. FIG. 3 shows a schematic of a workflow 300 related to the methods of the present invention. The present invention is not limited to the workflow 300 in FIG. 3.

With reference to the exemplary workflow 300 provided in FIG. 3B, step 302 provides for the extraction of DNA for a genetic sample. In step 304, a multiplex PCR is performed on 48 SNPs for the sample. In step 306, Illumina adapters are added to the genetic sample via PCR. In step 308, the sample is sequenced on a NGS such as an Illumina sequencer using, for example, 1×75 bp reads. In step 310, the reads from step 308 are aligned to the genome. In step 312, the number of reads supporting each allele are counted and the genotypes for the genetic sample are called. In step 314, sample swaps are identified by comparing the results of step 312, the called genotypes, to known genotypes, such as by comparing the called genotypes to reference sequences in a sequence library or database. In step 316, samples mixtures or contaminations are identified by identifying allele ratios and a mixture level is determined. In step 318, a sample status is identified based on at least the comparison in step 314 and identification in step 316, the sample status being, for example, one of clean (e.g., no contamination, mislabeling, or misidentification issues), mixture (e.g., contamination issues), or full swap (e.g., mislabeling or misidentification issues).

### Example 2

The following is a non-limiting example of the present invention. It is to be understood that said example is not intended to limit the present invention in any way. Equivalents or substitutes are within the scope of the present invention.

The following example describes an overview of particular embodiments of the methods and systems of the present invention.

DNA is extracted from collected sperm cells that have undergone processing and packaging into straws. The process below describes analyzing the DNA against known/reference DNA of the bull to help ensure that the DNA in the straw is the DNA of the bull printed on the straw. Likely contaminators are the analysis system's (algorithm's) best guess at a contaminator with the options being all bulls processed within a single run (e.g., in certain embodiments, if the true contaminator is not present in the sequencing run, it may not be identified). Note also that the sequencing run can comprise DNA from a variety of sources and may include conventional and sexed DNA. In certain embodiments, a full run is 96 DNA samples×6 plates. However, the present invention is not limited to these parameters.

DNA received from the production labs is amplified. The amplification process amplifies a set of SNPs, e.g., 48-49 SNPs, including known regions of variance. A unique profile of SNPs is known for bulls. The genotype for each parent animal may also be in possession. This provides a library of animal genotypes including a profile for each animal comprising the SNPs, e.g., 48-49 SNPs.

How the DNA reads (e.g., determine what the reads are) at each of the SNP locations is examined. Each of the DNA reads are compared to the library. First, there is an attempt to identify a 100% match. If a 100% match is identified, then the process may finish. If no 100% match can be identified, the system will try and identify combinations of known profiles in the library. The system can identify if the sample comprises, for example, Animal #1 plus something else (contamination from another sample). It may be determined that the sample is not what it was thought to be (e.g., an “alternative” or a sample swap or mislabeling), or it may be determined that the sample comprises some contaminant.

The estimation of a non-100% match may be based on, for example, a subset of 10-20 SNPs of the total target SNPs. The system can identify an approximate percentage mixture, e.g., +/−5%. In certain embodiments, the system can identify a mix and what the sample is mixed with. However, in certain embodiments, the system may identify a mix, but not necessarily what the sample is mixed with (e.g., it may not always be possible to identify the contaminator), e.g., if another individual is not available for comparison.

The sequencing data, e.g., NGS data, may be routed through to an internal database (e.g., an analysis system, e.g., application) from the sequencers. Data may be provided as an output in the app user interfaces. The data from the app may be exported as an output file. The output file may include all batches run and their standings. In some embodiments, the identity for each batch run is indicated in the output file and may include several columns or categories of additional information. For example, one category of information may be identity, wherein a “Pass” is when the identity is found and confirmed. Other categories of information may include: Purity; Genomics, wherein the information is related to whether or not the test sample is a mixture; Quantity, wherein the information is related to whether or not there was enough sample to run and analyze; Times, wherein the information is related to the number of times that animal's samples have been run (e.g., 32 of 32, which would mean that the animal's samples have been run 32 times and come back the correct each time; 155 of 167, which would mean that the animal's samples have been run 155 times but 12 times the sample came back mixed, etc.), etc. FIG. 5 shows a hypothetical example of the output file, wherein samples EX1-EX3 are flagged as potential mixes, wherein samples EX4-EX7 are shown to be matches. Samples EX1-EX3 are reviewed to determine the source of error. Samples EX4-EX7 pass all tests and are released for use.

As previously discussed, the thresholds for quantity and purity may be modified. Thus, the output data such as that in FIG. 5 is a function of the predetermined thresholds.

Manual reviews of items identified as mixed may be performed when: there is no genomic data to compare against, the sample is 7% mixed. In some embodiments, two straws are retested. If the same percentage of contamination and same number of contaminating SNPs are detected, the sample fails.

In certain embodiments, full swaps (e.g., mislabeling of a sample) may become a pass if appropriately identified to be another bull.

### Example 3

The following is a non-limiting example of the present invention. It is to be understood that said example is not intended to limit the present invention in any way. Equivalents or substitutes are within the scope of the present invention.

The following example describes an overview of the analysis system, e.g., application used for analysis and quality control. FIG. 4 shows a schematic view of the analysis system integrated with the sequencer and internal database, as well as quality control.

A user (e.g., technician) can log into the user interface of the analysis system (e.g., application). The user may use the system for reviewing certain samples, e.g., confirming genetic matches, determining whether or not a sample may have been a mixture, etc.

The analysis system may feature visual indicators that alert a user to a particular issue (e.g., possible mixture, lack of quantity, not a 100% match to expected reference animal, etc.).

A batch is called a run, and a run consists of several plates, each plate comprising several samples.

Failures may occur where there is not enough sequence information or not enough reads to make a determination about the purity or identity.

In certain instances, a technician will go through a list of mixtures. The results may be shown as a plot wherein the X axis is the loci (SNPs). If the sample is mixed, there will be a distinctive pattern (e.g., see FIG. 1A, FIG. 1B). In certain embodiments, SNPs that match consensus/known DNA of the individual bull may be labeled in a first design (e.g., whole, filled in circles; a first color, etc.); SNPs that do not match consensus/known DNA of the individual bull may be labeled in a second design (e.g., whole, empty circles; a second color; etc.); and SNPs that have no consensus/known DNA to compare to may be labeled in a third design (e.g., labeled with X; a third color; etc.). A clean sample will not have dots that are indicators of contaminations or unexpected results (e.g., red dots).

The analysis system, e.g., the plots, allows for determining when there has been an out of threshold mixture in the process, if the sample is potentially contaminated. The system helps visualize when the output of the process has gone wrong.

The analysis system may allow for a comparison to past samples. For example, a user may select recent runs to review. The system helps easily view outliers. The system may help identify mixtures (e.g., low level mixtures) occurring over a certain number of samples (straws), e.g., over 20 straws, 30, 40, 50, etc., which could indicate a problem in the process.

The system may allow for setting the thresholds for passing (e.g., identified as a genetic match), failing (e.g., identified as a mismatch or a possible mixture), and mixture (e.g., what is considered a mixture). In certain embodiments, the thresholds may be visualized by lines in the graphs or plots.

As previously discussed, the system may provide a means of indicating an issue such as contamination or too little DNA. In certain embodiments, the read counts and read percentile are indicators of how well the sequencing has performed, and the analysis system may provide confidence levels for the data.

As a non-limiting example, the system may use red dots on the plots as indicators of contaminations or unexpected results. FIG. 1C shows an example wherein the dots indicated as “evidence of mixing” are indicators of potential problems that require evaluation. For example, a heterozygous fail may be one wherein one of the mixes was not quite what was expected for the SNP; other fails may be shown as dots (e.g., red dots) in the middle of the plot or interface. In certain embodiments, the application allows for comparing the layered sample data to see areas of consistent failures for individual animals.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1.-22. (canceled)

23. A method of processing extracted DNA from one or more test samples to determine if the extracted DNA matches a known or reference DNA, said method comprising:
a. subjecting the extracted DNA from one of the one or more test samples to a nucleotide amplification using a one or more single nucleotide polymorphisms (SNP) primer pairs to generate a pool of SNP amplicons;
b. subjecting the pool of SNP amplicons to sequencing to generate a nucleotide sequence for each amplicon in the pool, wherein the pool of SNP amplicons includes known regions of variance, wherein a unique profile of SNPs is known for the reference DNA, wherein a genotype for each source of the reference DNA provides a library of source genotypes including the unique profile for each source comprising the SNPs;
c. comparing the nucleotide sequence of each SNP amplicon to each nucleotide sequence source genotype to identify a match or partial match, wherein:
a) if a 100% match is identified, then the extracted DNA matches a known or reference DNA; or
b) if the match is less than 100% match, then a combination of known profiles in the library is identified.

24. The method of claim 23, wherein the non-100% match is based on a subset of SNPs of a total target SNPs.

25. The method of claim 23, wherein an approximate percentage mixture is identified.

26. The method of claim 23, wherein the one or more test samples has a contaminant or is mislabeled.

27. The method of claim 23, wherein the extracted DNA comprises conventional and sexed DNA.

28. The method of claim 23, wherein a sequencing run comprises 96 DNA samples on 6 plates.

29. The method of claim 23, wherein amplification process generates 48 SNP amplicons.

30. The method of claim 23, wherein the nucleotide amplification is a multiplex assay using at least two or more SNP primer pairs.

31. The method of claim 23, wherein nucleotide amplification is a polymerase chain reaction (PCR) amplification and sequencing is next-generation sequencing (NGS).

32. A system for analyzing extracted DNA from one or more test samples, comprising:
a. a display;
b. a processor coupled to the display;
c. a memory coupled to the processor; and
d. a sequencer coupled to the processor;
wherein the memory stores an internal database and instructions that when executed by the processor, causes the processor to perform operations comprising:
i) subjecting the extracted DNA from the one or more the test samples to nucleotide amplification using one or more single nucleotide polymorphism (SNP) primer pairs to generate one or more SNP amplicons;
ii) subjecting the one or more SNP amplicons to sequencing, by the sequencer, to generate a nucleotide sequence for each of the one or more SNP amplicon,
iii) reviewing and analyzing the one or more test samples to confirm genetic matches and/or determine whether a sample is a mixture; and
iv) displaying results of said analysis on the display using visual indicators that alert a user to a particular issue.

33. The system of claim 32, wherein the results are plotted on a graph or plot wherein an X axis is the SNPs.

34. The system of claim 33, wherein the visual indicators of a mixed sample has a distinctive pattern that indicate contaminations or unexpected results.

35. The system of claim 33, wherein the system determines when there has been an out-of-threshold mixture.

36. The system of claim 33, wherein thresholds are set and visualized by lines in a graphs or plots.

37. The system of claim 32, wherein the memory stores results of past samples for use in comparison of current samples.

38. The system of claim 32, wherein read counts and read percentile are indicators of how well sequencing has performed.

39. The system of claim 33, wherein colored dots are used on plots as indicators of contaminations or unexpected results.
